(19) United States
(12) Patent Application Publication
(10) Pub. No.: US 2024/0130632 A1
(43) Pub. Date: Apr. 25, 2024

(54) SYSTEM FOR DETECTION OF VOLATILE ORGANIC COMPOUNDS (VOC) IN EXHALED BREATH FOR HEALTH MONITORING

(71) Applicant: KOZHNOSYS PRIVATE LIMITED, Pune (IN)

(72) Inventor: Jilma Peruvangat, Pune (IN)

(21) Appl. No.: 18/404,192

(22) Filed: Jan. 4, 2024

Related U.S. Application Data
(62) Division of application No. 17/272,966, filed on Mar. 3, 2021.

(30) Foreign Application Priority Data
Sep. 2, 2018 (IN) 201821016758

(51) Int. Cl. A61B 5/08; A61B 5/00; A61B 5/097; G01N 29/02; G01N 29/24

(52) U.S. Cl. CPC A61B 5/082; A61B 5/097; A61B 5/4845; G01N 29/022; G01N 29/2437; A61B 2562/046

(57) ABSTRACT

The present invention relates to a system for detection of volatile organic compounds (VOC) in exhaled breath for health monitoring. It can be used for screening, diagnosis, monitoring onset and relapse of diseases. Certain volatile compounds, in exhaled breath 5 of a person, which are the markers of the disease under consideration, will be analysed by the sensor array. The presence and concentration of these markers will be determined by a sensor array specific for the set of markers for the disease. Depending on the presence or absence and concentration levels of these VOCs health status of a person can be analysed. The present invention also relates to a device for monitoring health conditions of an 10 individual and screening for presence or relapse of diseases.

Cross reference to related application: The present application claims priority to Indian provisional patent application No 201821016758, filed on Sep. 3, 2018. The entire content of the aforementioned application is specifically incorporated herein by reference.

### FIELD OF INVENTION

The present invention relates to the field of health monitoring by analysis of exhaled breath sample. More particularly the present invention provides a device consisting of a sensor array for the screening of diseases and monitoring the health status by analyzing volatile markers present in the exhaled breath. The present invention also relates to a device for monitoring health conditions of an individual and screening for presence or relapse of diseases.

### BACKGROUND OF INVENTION

Health monitoring is very important to diagnose early onset of disease or for monitoring general health status of an individual. Breath test is a very easy way to do it as it is non-invasive and can be done any number of times as compared to blood tests.

Volatile compounds are present in breath as a result of metabolic processes within the body. Recent study has shown that many compounds in exhaled breath can serve as marker for diagnosis of diseases for example, ammonia for asthma, hydrogen disulphide for helicosis, ketones for cancer etc. If new compounds are present in breath it will be due to altered biochemical pathways as is in the case of many diseases. At times the concentration of compounds already occurring in exhaled breath also changes due to changes in metabolism associated with onset of disease. By determining the presence of these compounds or by analyzing changes in their concentrations in the breath the disease can be detected.

The most common practice of detecting diseases from exhaled breath involves Gas-Chromatography-Mass spectrometry. Disadvantages of using such devices are that it is very expensive and requires a trained professional for operating the device.

Accordingly there is a need for the sensor mechanism for detecting compounds in exhaled breath.

US20040166581A1 titled highly selective molecular sensor based on dual MIP/QCM elements and a method of use thereof filed on 26 Aug. 2004 discloses a molecular sensor for detecting small concentration of target molecules having similar shape and chemistry. Two QCM sensors are provided, both of which are covered with polymeric coatings having essentially the same chemistry. One of the QCM sensors is molecularly imprinted while the other is not. The output of the two QCM signals is compared to indicate the presence of the target molecule.

US 20120326092 A1 titled Volatile Organic Compounds As Diagnostic Markers For Various Types Of Cancer filed on 1 Jan. 2010 discloses sets of Volatile Organic Compounds (VOC) for breath analysis and method of diagnosing lung cancer by breath analysis methods of diagnosis, prognosis and monitoring of various types of cancer by determining the levels of signature sets of volatile organic compounds (VOCs) in a breath sample, wherein significantly different levels of said VOCs compared to a control sample are indicative for the presence of either one of breast, head and neck, prostate and colon cancers.

US20120326092 A1 discloses that the active area of the Quartz-Crystal Microbalance has to be in contact in liquid and is dependent on bodily fluids and doesn't teach about detection through exhaled breath.

There is a long felt need to develop alternate technologies for health monitoring, like breath analysis which is inexpensive, is easy to operate, gives sensitive and accurate results, displays results in few minutes. The present inventors have surprisingly developed a sensor and a device incorporating such a sensor which fulfills the aforesaid requirements and ameliorates the shortcomings of the prior art.

### OBJECTS OF THE INVENTION

It is an object of the present invention to provide a bench-top, portable, user friendly device for screening of diseases and monitoring of health by analysis of exhaled breath.

It is an object of the present invention to provide a medical device for screening presence, progress or relapse of disease with the help of exhaled breath of a person.

It is another object of the present invention to provide a mechanism for capturing and detection of volatile markers in exhaled breath of a person.

Yet another object of the present invention is to make a modular piezoelectric sensor array specific to volatile markers present in exhaled breath.

Still another object of the present invention is to make such sensors specific to volatile marker by coating said sensors with molecular imprinted polymer.

Another object of the present invention is to provide an array of such sensors arranged in a particular order.

Yet another object of the present invention is to provide a sensor array and breath collection chamber.

Yet another object of the present invention is to provide software control for working of the device, analyze the signal from the sensors and display the result.

Yet another object of the invention is to provide a control/display unit for controlling operation of the device and for displaying the results.

### SUMMARY OF THE INVENTION

According to an aspect of the present invention there is provided a molecularly imprinted polymer coated piezoelectric sensor array for detection of VOC from exhaled breath.

According to another aspect of the present invention there is provided a bench-top, portable device for the detection of VOC from exhaled breath.

According to yet another aspect of the present invention there is provided a method for detection of VOC from exhaled breath.

According to yet another aspect of the present invention there is provided a system for detection of VOC from exhaled breath.

### BRIEF DESCRIPTION OF THE DRAWINGS

The above and other aspects, features and advantages of certain exemplary embodiments of the present invention will be more apparent from the following description taken in conjunction with the accompanying drawings wherein:

FIG. 1 illustrates the assembly of the sensors

FIG. 2 illustrates the connectivity of the sensors with tubes

FIG. 3 illustrates a block diagram of a portable device (100) for detecting VOC from exhaled breath according to an embodiment of the invention.

FIG. 4 illustrates GC-MS spectra showing binding of volatiles on Non-imprinted polymer (NIP).

FIG. 5 illustrates GC-MS spectra showing binding of volatiles on toluene imprinted polymer (MIP) according to an embodiment of present invention.

FIG. 6 illustrates a flow chart for preparation of a MIP coated piezoelectric sensor according to an embodiment of present invention.

FIG. 7 illustrates frequency response of a toluene imprinted QCM sensor according to an embodiment of present invention.

### DETAILED DESCRIPTION OF THE INVENTION

The following description with reference to the accompanying drawings is provided to assist in a comprehensive understanding of exemplary embodiments of the invention. It includes various specific details to assist in that understanding but these are to be regarded as merely exemplary.

Accordingly, those of ordinary skill in the art will recognize that various changes and modifications of the embodiments described herein can be made without departing from the scope of the invention. In addition, descriptions of well-known functions and constructions are omitted for clarity and conciseness.

The terms and words used in the following description and claims are not limited to the bibliographical meanings, but, are merely used by the inventor to enable a clear and consistent understanding of the invention. Accordingly, it should be apparent to those skilled in the art that the following description of exemplary embodiments of the present invention are provided for illustration purpose only and not for the purpose of limiting the scope of the invention as defined by the appended claims and their equivalents.

It is to be understood that the singular forms “a,” “an,” and “the” include plural referents unless the context clearly dictates otherwise.

Features that are described and/or illustrated with respect to one embodiment may be used in the same way or in a similar way in one or more other embodiments and/or in combination with or instead of the features of the other embodiments.

It should be emphasized that the term “comprises/comprising” when used in this specification is taken to specify the presence of stated features, steps or components but does not preclude the presence or addition of one or more other features, steps, components or groups thereof.
The term “VOC” as used herein means Volatile organic compounds.
The term “MIP” as used herein means Molecular Imprinted Polymer.
The term “QCM” as used herein means Quartz Crystal Microbalance.

The present invention relates to a sensor for the detection of volatile markers present in the exhaled breath. The present invention also relates to a device and a system for monitoring health conditions of an individual and screening for presence or relapse of diseases.

The present invention provides a solution to the drawbacks associated with the prior art. Detection of the concentration of the volatile organic markers, which are present in the exhaled breath at different levels is with the help of a sensor array.

Present invention discloses a sensor mechanism for health monitoring using exhaled breath. This sensor mechanism forms a part of breath monitoring machine. A person whose health is to be monitored exhales into the machine for a certain period of time. The exhaled breath from the person is collected in a chamber which is attached to the sensor mechanism.

The said sensor is a piezoelectric sensor and has a particular resonant frequency when current is applied to it. The sensor is made specific by coating it with molecular imprinted polymer. Molecular imprinting is a technique of producing highly crosslinked polymers with complimentary cavities that binds to a chosen analyte molecule alone. Monomer, cross linker, initiator and template are added and polymerisation is carried out. The template molecules are the analyte molecules that are to be detected. After polymerisation is completed the template molecules are removed by washing or evaporation under elevated temperature. This leaves behind cavities in the polymer matrix that has affinity to bind to template molecules when they are present on the surface.

When the sensor is exposed to exhaled breath, the compound of interest binds to the imprinted polymer and there will be a change in frequency. This change in frequency will be proportional to the amount of the targeted compound present in breath. Thus the sensor allows specific detection of targeted volatile organic compounds in breath.

The molecularly imprinted polymer (MIP) coated piezoelectric sensor for detection of VOCs of the present invention comprises:
a) A polymer film or polymer nanoparticles molecularly imprinted with the VOC target molecule and coated on;
b) A piezoelectric crystal having frequency sensitive to binding with the said VOC target molecule, and
c) Interdigitated electrodes, located on a surface of the piezoelectric crystal coated with imprinted polymer film or nanoparticles, for measuring a change in the frequency to sense said VOC binding.

In an embodiment, the molecularly imprinted polymer layer comprises a polymer synthesized in the laboratory. The molecular imprinted polymer is composed of monomers selected from the group and not limited to Acrolein, Acrylamide,2-Acrylamido-2-methylpropane sulfonic acid (AMPSA), Acrylic acid,Acrylonitrile,Allylamine,m-divinylbenzene(DVB),p-divinylbenzene(DVB), N,N-Dimethyl Aminoethyl Methacrylate,Ethylene glycol dimethacrylate, 2 hydroxyethyl methacrylate (HEMA), Itaconic acid, Methacrylic acid, N,N′-Methylenebisacrylamide,Urocanic acid, Urocanic acid, ethyl ester, vinylbenzene, 1-Vinylimidazole, 2-Vinylpyridine, 4-Vinylpyridine, 2-(Trifluoromethyl)acrylic acid, 4-Vinylbenzoic acid, 4-vinylbenzeneboronic acid, N-vinylpyrrolidone (NVP), Methyl methacrylate, Acrylonitrile or combinations thereof.

In an embodiment, the crosslinker is selected from the group and not limited to Ethylene glycol dimethacrylate(EGDMA),m-divinylbenzene(DVB),p-divinylbenzene(DVB),N,O-bismethacryloyl,ethanolamine,N,N′-methylenebisacrylamide (MDAA),p-divinylbenzene(DVB),N,N′-1,3-phenylenebis(2-methyl-2propenamide) (PDBMP)3,5-bisacryloylamidobenzoicacid,N,O-bisacryloyl-Lphenylalaninol,1,3-diisopropenylbenzene (DIP),pentaerythritol triacrylate (PETRA),pentaerythritol pentacrylate (PRTEA), triethylolpropane, trimethacrylate (TRIM), tetramethylenedimethacrylate (TDMA), 2,6-bisacryloylamidopyridine,1,4-phenylenediacrylamide,1,4-diacryloyl piperazine (DAP), N,N′-ethylene bismethacrylamide,N,N′-tetramethylene, bismethacrylamide, N,N′-hexamethylene, bismethacrylamide, anhydroerythritol dimethacrylate, Ag—LaFeO3 or 1,4;3,6-dianhydro-Dsorbitol-2,5-dimethacrylate.

In an embodiment, the nanoparticles have a particle size ranging from 100 to 500 nm.

In an embodiment, the piezoelectric sensor is selected from and not limited to micro/nanocantilever, quartz crystal microbalance or surface acoustic wave sensors.

In an embodiment, the sensor detects target VOCs selected from ammonia, hydrogen disulphide, ketones, Benzene, Ethylbenzene,4-ethylbenzamaide, undecanal, diethyl carbitol, isoborneol, n-propylbenzene, 1-Butanol, 2-Butanone, 2-Pentanone, n-pentane, n-hexane, n-heptane, n-octane, n-dodecane, 2-Methylpentane, 3-Methylpentane, Cyclohexane, Propanal, n-butanal, n-pentanal, n-hexanal, n-octanal, n-nonanal, n-decanal, Naphthalene, 1-methyl-, Cyclohexane 1,4-dimethyl, Cyclohexane, 1,3-dimethyl-trans-, Cyclohexane, 1-ethyl-2-methyl-trans-, Heptane, 3-ethyl-2-methyl-, Benzene, 1,2,3,4-tetramethyl-, Benzene,1,4-dichloro-, 2-Propanol, 1-Propanol, 2,2-dimethyl, n-hexanol, Methylene chloride, Styrene, Tetrachloroethylene, Toluene, m,p-Xylene, o-Xylene, p-dichlorobenzene, 4,6-Dimethyl-dodecane, 2,2-Dimethyl-propanoic acid, 5-Methyl-3-hexanone, 2,2-Dimethyl-decane, Limonene, 2,2,3-Erimethyl-,exo-bicyclo[2.2.1]heptane, Ammonium acetate, 3-Methyl-hexane, 2,4-Dimethyl-heptane, 4-Methyl-octane, 2,6,6-Trimethyl-octane, 3-Methyl-nonane, 2,3-dihydro-1-phenyl-4(1-H)-quinazolinone, 1-Phenyl-ethanone, Heptanal, Isopropyl myristate, Hydrazine-carboxamide, Methyl hydrazine, Ethyl alcohol, 1-Methyl-4-(1-methylethyl)-benzene, Dimethyl ether, Butylated hydroxytoluene, Carbonic dihydrazide, 1-Methyl-2-(1-methylethyl)-benzene, 1-Methyl-3-(1-methylethyl)-benzene, 1,2,3-Cycloheptatriene, 3-Ethyl-pentane, 1,3,5,7-Cyclooctatetraene, Bicyclo [4.2.0]octa-1,3,5-triene, 2,3,4-Trimethyl-hexane, 2,6-Bis(1,1-dimethylethyl)-4-methyl-methylcarbamate, phenol, 4,7-Dimethyl-undecane, 2,4,6-Tris(1,1-dimethyl-ethyl)-4-methylcyclohexa-2,5-dien-1-one, Hydrazine, 1,3-Pentadiene, 3,3-Dimethyl-pentane, 3,3-Dimethyl-hexane, 2-Methyl-hexane, 3-Ethyl-hexane, 2,2,3-Trimethyl-hexane, Ethylidene cyclopropane, 2-Ethyl-1-hexanol, 2-Ethyl-4-methyl-1-pentanol, 2,3,4-Trimethyl-pentane, 2,3-Dimethyl-hexane, 3-Ethyl-3-methyl-2-pentanone, 2-Methyl-4,6-octadiyn-3-one, 2-Propyl-1-pentanol, 6,10-Dimethyl-5,9-dodecadien-2-one, 2-propenenitrile, 2-butoxy-ethanol, furfural, 6-Methyl-5-hepten-2-one, Isoprene, 1,2-Propanediol, 2-Acetyl aminopropionic acid, Cyclopentanone, Methylacrylic acid, Butyl acetate, Trans-2-Butene Oxide, Dimethylacetamide, Benzocyclobutene, Cyclohexanone, Butyl Glycol, 4-Hydroxybutanoic acid, 1,3,5,7-Tetroxane, Ethylene Carbonate, 1,4-Dimethoxy-2,3-butanediol, 2,5,6-Trimethyl-octane, 3,4,5,6-Tetramethyloctane, 2,3,4-Trimethyl-heptane, 5-Methyl-3-hexanol, 5-Butylnonane, 2,3,6-Trimethyl-octane, Benzenemethanol, alpha, alpha-dimethyl, Ethylaniline, Cyclooctanemethanol, trans-2-Dodecen-1-ol, 2,5-Dimethylhexane-2,5-dihydroperoxide, Tetradecane, Hexadecane, butane,2-methyl, Ethanol, Acetone, Isopropyl alcohol, Acrolein, Furan, tetrahydro-, Heptane, Carene, Tetradecanal, 3,3-dimethylhex-1-ene, 2-buten-1-ol, N-methyl-2-methylpropylamine, n-octene, Benzothiazole, Propane,2-methyl-, 1,3-Butadiene, Acetonitrile, n-Butane, Furan, Dimethyl sulphide, 2-Pentene, (Z)-, 1,3-Pentadiene, (E)-, 1,3-Pentadiene, (Z)-, 2-Propenal, 2-methyl-, 3-Buten-2-one, Furan, 2-methyl-, 2,3-Butanedione, Furan, 3-methyl-, Ethyl acetate, Thiophene, Pentane, 2-methyl-, 1-Pentene, 2-methyl-, 1-Hexene, Pentane, 3-methyl-, Pyrrole, Pyrimidine, Furan, 2,5-dimethyl-, Sulfide, allyl methyl (ams), Pyridine, Sulfide, methyl propyl (mps), Hexane, 2-methyl-, 1-Heptene, 2-Heptene, (E)-, 2-Hexanone, Heptane, 2-methylene, 3-Octene, (E)-, 2-Cyclohexen-1-one, 4-Heptanone, 3-Heptanone, 2-Heptanone, Heptane, 2,4-dimethyl-, -Pinene, Furan, 2-pentyl-, 3-Carene, m-Cymene, p-Cymene, Eucalyptol, Pyrrolidine, 2,4-Dimethyl-1-heptene, 2,2-Dimethyl-butane, 1,3-Di-tert-butylbenzene, 2-Xylene, 2-Nonanone, 4-Methyl-2-heptanone, 2-Dodecanone, Isobutyric acid, allyl ester, 2-Ethyl-hexanol, Benzaldehyde, Cyclohexanol, 3-methylbutanal, Propanoic acid, Octane, Terpene,1-hexadecanol, Dimethyl disulphide, Xylene, Ethane, Propane, Methanol, 2,3,4-Trimethylhexane, 2,6,8-Trimethyldecane, Tridecane, Undecane, 2,4-Dimethylheptane, 4-Methyloctane, 2,2,4-Trimethylhexane, Decane, 3,3-Dimethyl-heptane, 2,4-Dimethyl-octane, 3-Ethyl-3-methyl-heptane, 2,3,7-Trimethyl-decane, 2,3-Dimethyl-decane, 3,9-Dimethyl-undecane, 3,6-Dimethyl-decane, 2,5,6-Trimethyl-decane, Tetradecane, Pentadecane, 2-Methyl-tridecane, 2-Methyl-pentadecane, Acetophenone, Acetic Acid, 2-Methyl-1,3-butadiene, Phenylethyl acetate, Phenol, Carbon dioxide, Nonadecane, Phthalic anhydride, Sulphur dioxide, Acetaldehyde, Acrylonitrile, Carbon disulphide, 1-Decene, 1-Nonene, 1-Octene, 3-Methyl-hexane, (E)-2-Nonene, Ammonia, Hydrogen sulphide, Triethyl amine, Trimethyl amine, 2,6,10-Trimethyldodecane, 3,7-Dimethyl-decane, 2,3-Dimethyl-heptane, 2,2,4,6,6-Pentamethyl-heptane, 5-Ethyl-2-methyl-octane, 2,6,10,14-Tetramethyl-, hexadecane, 3,7-Dimethyl-propanoate(E)-2,6-octadien-1-ol, 2,3,5-Trimethyl-hexane, (1-methylethyl)benzene, (1-methylpropyl) cyclooctane, 2-Ethylhexyl tetradecyl ester oxalic acid, 2-Butyl-1-octanol, 1-Chloro-nonadecane, 3-Ethyl-2,2-dimethyl-pentane, 1,1′-oxybis-octane, 2,3,6,7-Tetramethyl-octane, Decamethyl-cyclopentasiloxane, 1-Propanol, Methanethiol, 2-Butene, Isobutane, 2-Methyl-1-propene, Pentafluoroethane, Ethyl ether, Methyl acetate, 2,3-Butadiene, Dichlorofluoroethane, 2-Methylbutane, 2-Pentene, Trichloro-methane, Cyclopentane, 2-Methylpropanal, 2-Methylfuran, 2-Methyl-1-pentene, 2-Methyl-1-propanol, Methylcyclopentane, Mercaptoacetone, 2-Ethoxy-2-methylpropane, 1-Pentanol, 4-Methylpentane, Methylcyclohexane, Tetrahydro-2,2,4,4-tetramethylfuran, Nonane, 3,5-Dimethyloctane, 3-Ethyloctane or combinations thereof.

### Sensor Array:

The present invention also provides a sensor array for detection of VOCs from exhaled breath comprising:
i. One or more molecularly imprinted polymer (MIP) coated piezoelectric sensor comprising a polymer film or polymer nanoparticles molecularly imprinted with the VOC target molecule and coated on; a piezoelectric sensor having frequency change sensitive to binding with the said VOC target molecule, and interdigitated electrodes, for measuring a change frequency to sense said VOC binding;
ii. A reference sensor comprising a non-imprinted polymer film or nanoparticles or a non-polymer film on; a piezoelectric sensor and interdigitated electrodes, located on the piezoelectric sensor, for measuring a change in the frequency;
   Wherein, the said sensor array is configured to quantify the difference between the frequency of the MIP sensor and the frequency of the reference sensor to determine a concentration of the target VOC molecule.

Sensor array consists of plurality of active sensors and reference sensors. Each active sensor is individually specific for detection of a single targeted compound. The plurality of reference sensors will either be a polymer coated, non-coated or both. The frequency of the plurality of active sensors will change in the presence of certain volatile organic compound. As the volatile marker binds to the MIP on the plurality of active sensors, the frequency of the active sensor changes and reaches equilibrium. The frequency of the plurality of reference sensors also changes due to the presence of moisture and other compounds in breath. These frequency changes are noted by the frequency counters connected to the each of the plurality of active and reference sensors. The frequency change of active sensors and reference sensors is then compared and a resultant frequency is obtained. This resultant frequency will be directly proportional to the concentration of volatile markers.

The sensor system has two phases' viz. calibration phase and detection phase. These phases are controlled automatically by software in the breath monitoring machine or controlled manually by the operator. Each cycle of breath monitoring starts with a calibration phase. During calibration the said sensor array is flushed with heated nitrogen or air. This causes the bound VOCs from earlier cycle to dissociate from the sensor, making it ready for next detection cycle. At the end of calibration phase the frequency of the active sensors and reference sensors are noted. During detection phase the exhaled breath from the breath collection chamber is passed through the sensor chamber. The frequency change of each of the plurality of active sensor is noted using frequency counters and compared with plurality of reference sensors to get the resultant frequency. The correlation of frequency changes and concentration of markers are determined by the system. The health status of the individual will be displayed on the screen of breath monitoring machine based on the software analysis of frequency changes of the sensor.

The sensor assembly is connected with the help of tubes with the controlling valves which acts as breath carrier as well. The tubes are made of non-reactive material selected from Teflon, Silicon or Tygon. The connection or arrangement of the tubes is such that the flow of the breath will be at an angle to the surface of the sensor. The tubes are connected with the sensors in such a way that outflow of a sensor acts as the inflow of adjacent sensor in the sensor assembly connected in series.

### Device

The present invention also provides a device that analyses particular compounds in exhaled breath for detection and monitoring of diseases. The device tests the exhaled breath for presence of particular compounds and their concentration and analyses the health status of an individual. This device can be used as a screening device for detection of diseases or group of diseases or as a device for monitoring the progress of a disease or group of diseases.

In an embodiment, the present invention provides a bench top, stand-alone and portable device and a method for analysis of exhaled breath and early detection of certain diseases and monitoring the progress of certain diseases. The device analyses breath in a few minutes and displays the results on screen immediately. The device can be operated by people with minimum skill and the results can be interpreted easily by a non-medical person also.

The bench top, stand-alone and portable device of the present invention comprises the following components:
i. An inlet port for collecting exhaled breath;
ii. A pre-concentrator connected to receive the exhaled breath sample from the sample inlet port to form a concentrated sample;
iii. A heating unit arranged for desorbing the captured VOCs from the adsorbent material of the pre-concentrator;
iv. A sensor chamber connected to receive the concentrated sample from the pre-concentrator and configured to detect and quantify VOCs therein; Wherein the sensor chamber comprises a sensor array comprising a plurality of molecularly imprinted polymer (MIP) coated piezoelectric sensor for detecting the VOCs and a non-imprinted polymer film or a non-polymer film coated reference sensor;
v. A gas handling system for transporting the sample from the sample inlet port to the pre-concentrator and the concentrated sample from the pre-concentrator to the sensor array and from the sensor array to an outlet;
vi. A software algorithm to analyse and process signals; and
vii. A control unit for controlling operation of the device and a output unit for displaying the results.

In an embodiment, the pre-concentrator comprises an adsorbent material for reversibly capturing the VOCs of exhaled breath and removing carbon dioxide, moisture and other unwanted constituents of exhaled breath.

In an embodiment, the adsorbent is selected from molecular imprinted polymer, polymer resins, activated charcoal, divinylbenzene, polydimethylsiloxane, polyacrylate, polyethylene glycol or graphitized carbon black.

In an embodiment, the pre-concentrator is connected to a carbon-dioxide sensor for determining the carbon-dioxide in the exhaled breath

In an embodiment, the gas handling system includes an air intake port to purge the Pre-concentrator with dry air. In an embodiment, the air intake port is connected to an air filter.

In an embodiment, the gas handling system includes a flow sensor connected to the sample inlet and sensor chamber and means to select a desired portion of a stream of breath exhaled into the sample inlet.

In an embodiment, the gas handling system comprises plurality of valves.

In an embodiment, the device comprises temperature sensors for sensing temperature of the air in pre-concentrator.

In an embodiment, the inlet port is adapted to receive exhaled breath directly from the subject by the subject exhaling into the inlet.

In another embodiment, the sample inlet is adapted to receive exhaled breath from a receptacle.

In an embodiment, the sensor array is modular. Sensor array consist of multiple sensors each specific for sensing multiple VOC markers of the targeted disease and a reference sensor.

The device can detect or monitor different diseases by changing the sensor module. The device has a different sensor module for different diseases. Furthermore, sensors can be reused

The device has a pump that draws air through the device and its various units

The device has a number of electronic valves and connectors to ensure leak proof transfer of airstream to different units of the device

The device has a software algorithm to process signal from sensor array

The device has a display which allows user to control the device. The device has a display to show breath analysis result

In an embodiment, the device has memory for storing reports of at least 100 patients

The device 3 cycles for analysis of exhaled breath volatiles:
A. Warm-up cycle: heated gas passes through the pre-concentrator and sensor chambers to flush the MIP of adsorbed VOCs from previous cycle;
B. Breathing cycle: where one person breathes normally and the exhaled portion of breath is passed through pre-concentrator for adsorption of VOC;
C. Analysis phase: where pre-concentrator is heated and the adsorbed VOCs are desorbed from the adsorbent material and is carried by air stream to the sensor chamber. The VOCs adsorb only to the specific sensor and generates a signal.

### Method and System:

The present invention provides a method for detecting and quantifying volatile organic compounds in breath using the device of the present invention comprising the steps of:
I. Exhaling into an inlet port for collecting exhaled breath;
II. Directing the exhaled breath to the pre-concentrator while heating the pre-concentrator to a first temperature;
III. Purging the pre-concentrator with dry air;
IV. Sealing the pre-concentrator and heating it to a second temperature higher than the first temperature to release volatile organic compounds;
V. Passing the released volatile organic compounds to the sensor chamber to detect and quantify the volatile organic compounds; and
VI. Purging the pre-concentrator while heating it to an elevated temperature to remove any remaining volatile organic compounds.

In an embodiment, the method further comprises the step, before and/or after analyzing the concentrated sample, of controlling the gas handling system to admit ambient air into the sensor chamber for calibration.

Advantages of the present invention are:
1—Sensor allows development of a new breath based assay for health monitoring
2—Allows for development of a low cost technique for disease screening
3—This sensor allows specific detection of volatile organic compounds in exhaled breath of individual, pertaining to different diseases.
4—This leads to less false positive readings and also increases the sensitivity for detecting diseases at early stages.
5—The present invention provides a bench-top, stand-alone device to analyze breath in a few minutes and displays the results on screen immediately.
6—The device uses a combination of sensors, pre-concentrator, software and other units in the device.
7—Capability of the device for detecting multiple diseases at one time.
8—Sensor module which can be replaced.
9—The device can be operated by people with minimum skill and the results can be interpreted easily by a non-medical person also.

Reference is now made to FIGS. 1 to 6 which illustrate the various embodiments of the present invention. The examples are presented to exemplify the invention and are not to be considered as limiting the scope of the invention.

Reference numbers of the components according to various embodiments of the present invention are listed below for ready reference:

| Reference No | Name of the component |
|---|---|
| 1 | Tubes to carry the breath for analysis |
| 2 | Sensor array comprising of reference and |
| | active sensors |
| 3 | Frequency counters for measuring the |
| | frequency |
| 4 | Processor for processing the signals |
| 5 | Display for displaying the results |
| 10 | Sensors |
| 11 | Inlet to sensor array |
| 12 | Outlet of 1^{st} sensor in array |
| 13 | Inlet of 2^{nd} sensor |
| 14 | Outlet of sensor array |
| 100 | Device |
| 101 | inlet port |
| 102 | pre-concentrator |
| 103 | heating unit |
| 104 | sensor chamber |
| 105 | gas handling system |
| 106 | outlet pump |
| 107 | exhaust |
| 108 | control unit (electronics main board) |
| 109 | output unit (display touch screen) |
| 110 | carbon-dioxide sensor |
| 111 | air intake port |
| 112 | air filter |
| 113a, 113b | Flow sensors |
| 114a, 114b, | solenoid valves |
| 114c and 114d | |
| 115a, 115b | Temperature sensors |

FIG. 1 illustrates the assembly of the sensors according to an embodiment of the present invention.

A person's breath is carried through a non-reactive tubing (1) to the sensor array (2) during detection phase. These tubes (1) also carry the nitrogen or air for cleaning the sensors during calibration phase. The sensor array (2) has reference sensors and active sensors arranged in series. The breath from tubing (1) first enters the reference sensor and is then carried to the active sensors and flushed out of the monitoring device. Each of the reference sensors and active sensors are connected to a frequency counter (3) that calculates the frequency of each of these sensors in real time. The frequency from the counters (3) are processed using a main computer (4), which has the software algorithm that co relates the frequency of active sensors to concentration of markers and disease. After processing, the results are displayed on the device (5) which shows concentration of each marker and disease associated with it.

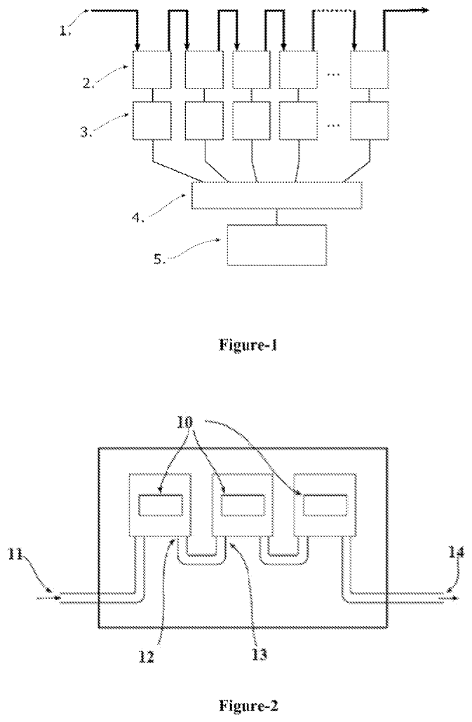

FIG. 2 illustrates the connectivity of the sensors with tubes in an embodiment according to present invention

Sensing element (10) consists of a number reference sensors and active sensors. The active sensors are coated with different molecular imprinted polymers for detection of different markers. The reference sensors are non-coated or coated with non-imprinted polymer. The tubes carrying the breath enter the reference sensors first and then the active sensors. The air inlet tubing (11) on the sensor assembly is directly pointed to the MIP coated area of the sensor (10) and the direction of flow of the breath is perpendicular to the sensor. The breath interacts with the reference sensor and the moisture and other compounds in breath binds to the sensor creating a frequency change. Remaining breath from the reference sensor is carried to the active sensor through the outlet port from reference sensor (12). This outlet port (12) acts as the inlet port (13) of active sensor. The VOCs in breath selectively binds to MIP on the sensor and the frequency change is noted. After the VOCs in breath binds to the MIP the remaining breath is flushed out of the device through the tubing through air exhaust port (14).

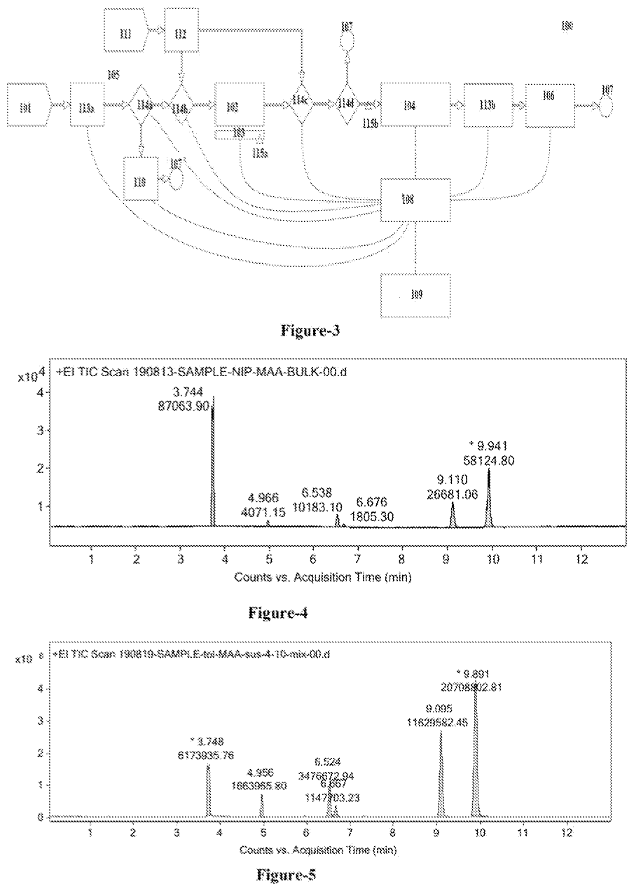

FIG. 3 is a block diagram of a portable device (100) for detecting VOC from exhaled breath according to an embodiment of the invention. Dashed lines specify electrical connections and double lines with arrow heads indicate flow-path

The device (100) comprises:
An inlet port (101) for collecting exhaled breath;
A pre-concentrator (102) connected to receive the exhaled breath sample from the sample inlet port (101) to form a concentrated sample. In an embodiment, the pre-concentrator (102) is connected to a carbon-dioxide sensor (110) for determining the carbon-dioxide in the exhaled breath
A heating unit (103) arranged for desorbing the captured VOCs from the adsorbent material of the pre-concentrator (102);
A sensor chamber (104) connected to receive the concentrated sample from the pre-concentrator (102) and configured to detect and quantify VOCs therein; Wherein the sensor chamber (104) comprises a sensor array comprising a plurality of molecularly imprinted polymer (MIP) coated piezoelectric sensor for detecting the VOCs and a non-imprinted polymer film or nanoparticle or a non-coated reference sensor as shown in FIG. 2.
A gas handling system (105) for transporting the sample from the sample inlet port to the pre-concentrator (102) and the concentrated sample from the pre-concentrator (102) to the sensor chamber (104) and from the sensor chamber (104) to an outlet pump (106) and then exhaust (107);
   The gas handling system (105) includes an air intake port (111) to purge the Pre-concentrator (102) with dry air. The air intake port is connected to an air filter (112) for filtering the air to remove unwanted particles before entering the pre-concentrator (102).
   The gas handling system (105) also includes a flow sensors (113a, 113b) connected to the sample inlet (101) and sensor chamber (104) and means to select a desired portion of a stream of breath exhaled into the sample inlet (101).
   The gas handling system comprises a plurality of valves. In this device, the valves are solenoid valves (114a, 114b, 114c and 114d)
A control unit (108) for controlling operation of the device (100) and an output unit (109) for displaying the results. The output unit (109) may be a display touchscreen.

The device (100) also comprises temperature sensors (115a, 115b) for sensing temperature of the air in pre-concentrator.

### Device Operation:

The device (100) has three cycles a) Warm-up cycle b) breathing cycle c) analysis cycle.

During the breathing cycle, the individual exhales in to the inlet port (101) of the device (100) using a mask (not shown). The mask is connected to bacterial filter and moisture filter (not shown). The mask has tube connected to the inlet port (101) of the device. The inlet port (101) of the device is in turn connected to an on/off valve, to facilitate optimal breath collection. When a person exhales the breath passes through the bacterial and moisture filter and enters the inlet port (101). One end of inlet port (101) is connected to the filter and the other end is connected to a solenoid valve (114a). The solenoid valve (114a) receives signal for switching on and off from carbon-dioxide sensor (110)/flow sensor (113a).

The inlet port (101) has a bifurcating tube going to a carbon-dioxide sensor (110) or a flow sensor (113a). The carbon-dioxide sensor (110) analyses the amount of carbon-dioxide present in the exhaled breath. When the amount of carbon-dioxide detected is above a certain threshold, it sends signal to a solenoid valve (114a), which opens the end of the inlet valve (114b) connected to pre-concentration unit (102). The pre-concentrating unit (102) consists of either a commercially available adsorption material like polymer resins, activated charcoal, divinylbenzene, polydimethylsiloxane, polyacrylate, polyethylene glycol or graphitized carbon black etc or custom made molecular imprinted polymer cartridge. When the solenoid valve (114b) is open, the exhaled breath of the person passes through the pre-concentrating unit (102) and the VOCs are captured in the adsorption material packed column. A person breathes normally for a few minutes allowing the adsorbent material to be saturated with the VOCs from the person's breath.

After completion of the breathing cycle the machine goes into analysis cycle. During this cycle the solenoid valve (114c) is shut. The solenoid valve (114b) is open which allows air from outside to enter through an air filter (112) and pass through the adsorbent material. The adsorbent material is heated to temperature up to 300° C. which causes all the VOCs adsorbed to desorb from the column. These desorbed VOCs are carried by a stream of air to the sensor chamber (104).

The sensor chamber (104) consists of an array of sensors as shown in FIG. 2. These sensors are sealed. Each sensor chamber however has an inlet and outlet for air stream to pass over the sensor surface, which allows interaction of the VOCs in airstream to the sensor coating. The sensors are piezoelectric crystals with their surface coated with molecular imprinted polymer. This molecular imprinted polymer is made specific for the disease VOC markers we want to detect. When markers of a particular disease are present in the exhaled breath of a person it binds to the pores of the MIP. As a result frequency of the piezoelectric crystal changes. This change in frequency is proportional to concentration of the VOC being bound to the MIP. So by calculating the change in frequency the concentration of the particular VOC in breath is calculated. Also each sensor binds to the VOC it is meant to detect owing to the specific nature of the MIP coating.

The warm-up cycle consists of passing a heated airstream through the pre-concentrator (102) and the sensor chamber (104) so as to dissociate the remaining VOCs. After the calibration cycle the sensor is ready to be used for the next persons breath testing.

### EXAMPLES

The following examples are meant to illustrate the present invention. The examples are presented to exemplify the invention and are not to be considered as limiting the scope of the invention.

### Example 1

### Toluene Imprinted Polymer

Non-imprinted polymer (control/reference) synthesised was exposed to a volatile gas mixture containing 1000 ppm of Acetone, Isopropanol, Methanol, Ethanol, Tricholoromethane and Toluene. The binding of these volatiles were analysed using Gas-Chromatography Mass Spectrometry (FIG. 4, Table 1 below)

TABLE 1

| | | Retention | | |
|---|---|---|---|---|
| Peak | Compound Name | Time | Area | Area % |
| 1 | Nitrogen | 3.744 | 87063.9 | 100 |
| 2 | Acetone | 4.966 | 4071.15 | 4.68 |
| 3 | Isopropanol | 6.538 | 10183.1 | 11.7 |
| 4 | Ethanol | 6.676 | 1805.3 | 2.07 |
| 5 | Tricholoromethane | 9.11 | 26681.06 | 30.65 |
| 6 | Toluene | 9.941 | 58124.8 | 66.76 |

Toluene imprinted polymer synthesised was exposed to a volatile gas mixture containing 1000 ppm of Acetone, Isopropanol, Methanol, Ethanol, Tricholoromethane and Toluene. The binding of these volatiles were analysed using Gas-Chromatography Mass Spectrometry (FIG. 5, Table 2 below).

TABLE 2

| | | Retention | | |
|---|---|---|---|---|
| Peak | Compound Name | Time | Area | Area % |
| 1 | Nitrogen | 3.748 | 6173935.76 | 29.81 |
| 2 | Acetone | 4.956 | 1663965.8 | 8.04 |
| 3 | Isopropanol | 6.524 | 3476672.94 | 16.79 |
| 4 | Ethanol | 6.667 | 1147703.23 | 5.54 |
| 5 | Tricholoromethane | 9.095 | 11629582.45 | 56.16 |
| 6 | Toluene | 9.891 | 20708802.81 | 100 |

It is observed from the aforesaid results that toluene imprinted polymer binds more to toluene compared to other volatiles. This shows that toluene imprinted polymer has specific binding sites for toluene.

The aforesaid data clearly demonstrates that the toluene imprinted polymer of the present invention has specificity and doesn't bind with other volatiles. The data provided is a qualitative experimental data for molecular imprinting capacity of the MIPS synthesized.

### Example 2

### Preparation of the MIP Coated Piezoelectric Sensor

Molecular imprinted polymer is produced by mixing monomer, crosslinker and template in particular ratio. The monomer, crosslinker and template is added to a solvent a mixed well. Nitrogen is purged through the mixture so make and inert environment for the reaction to initiate. Initiator is added to the mixture and heated. Reaction is allowed to proceed till it forms a bulk polymer, or after it reaches gel point sodium dodecylsulphate and water is added and stirrer for 24 hours to from nanoparticles of imprinted polymer.

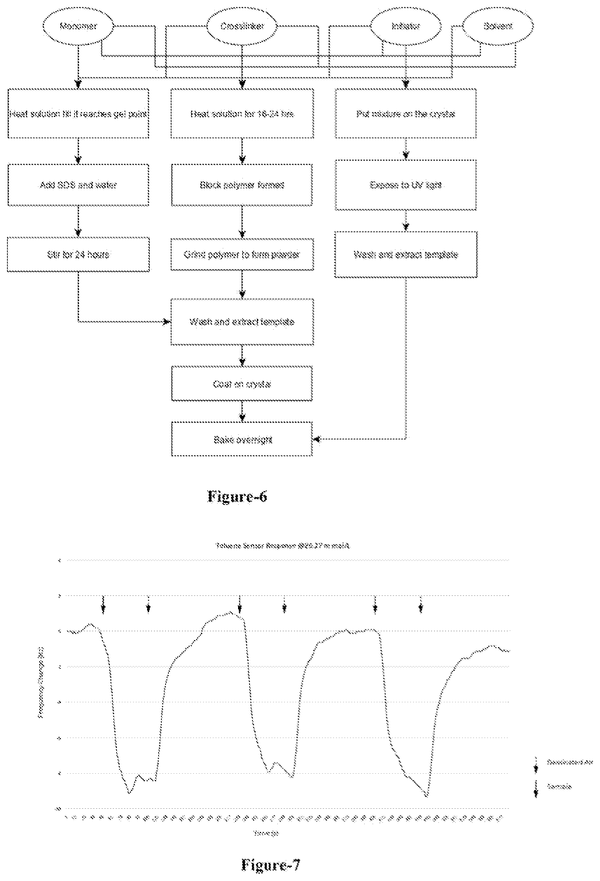

To coat thin film on piezoelectric crystal the mixture is directly coated on the crystal and the reaction takes place in the presence of UV light. After formation of imprinted polymer, the template is extracted by washing the polymer. The polymeric nano particles are coated on the piezoelectric crystal and baked over night before using the sensor for measurements. Flow chart for preparation of sensors is shown in FIG. 6.

In Example 1 and Example 2, toluene imprinted polymers were prepared by using Methacrylic acid and Ethylene glycol dimethacrylate (EGDMA), using toluene as solvent. The toluene imprinted nanoparticles were then coated on quartz crystal microbalance as in Example-2.

### Sensor Characterization:

The frequency response of the toluene imprinted QCM sensor prepared in above Example-2 was studied.

FIG. 7 illustrates the change in frequency of toluene imprinted QCM sensor when 26.27 mmol/L of toluene was passed through the sensor chamber.

It is to be understood that the present invention is susceptible to modifications, changes and adaptations by those skilled in the art. Such modifications, changes, adaptations are intended to be within the scope of the present invention.

1-5. (canceled)

6: A modular sensor array for detection of VOCs from exhaled breath comprising:
i. One or more molecularly imprinted polymer (MIP) coated piezoelectric sensor comprising a polymer film or polymeric nanoparticle molecularly imprinted with the VOC target molecule and coated on; a piezoelectric sensor having frequency sensitive to binding with the said VOC target molecule, and
ii. A reference sensor comprising a non-polymer or non-imprinted polymer film coated on; a piezoelectric sensor, for measuring a change in the frequency;
Wherein, the said sensor array is configured to quantify the difference between the frequency of the MIP sensors and the frequency of the reference sensor to determine a concentration of the target VOC molecule.

7-25. (canceled)
